(19) United States
(12) Patent Application Publication
(10) Pub. No.: US 2026/0305572 A1
(43) Pub. Date: Oct. 8, 2026

(54) HOOF BOOT WITH A REACTION-FORCE MEASURING PLATE

(71) Applicant: CONTITECH DEUTSCHLAND GMBH, Hannover (DE)

(72) Inventors: Max Konstantin WERHAHN, Hannover (DE); Peter Michael MARIENFELD, Hannover (DE)

(21) Appl. No.: 19/474,778

(22) PCT Filed: Mar. 27, 2024
(86) PCT No.: PCT/EP2024/058210

§ 371 Date: Oct. 13, 2025

(30) Foreign Application Priority Data
Apr. 13, 2023 (DE) 10 2023 203 355.0

(51) Int. Cl. A01K 13/00; A01K 29/00; A61D 99/00

(52) U.S. Cl. CPC A01K 13/007; A01K 29/005; A61D 99/00

(57) ABSTRACT

The present invention relates to a hoof boot (15) with a reaction force measuring plate (1) for detecting the distribution of ground reaction forces across the ground contact area of the foot of a hoofed animal when it strikes the ground, with a preferably rigid carrier plate (3) with a first surface (3a) facing the hoof (H) during use and an opposite second surface (3b) facing the ground, a plurality of flat force measurement sensors (5) fixed in position on the first surface (3a) of the carrier plate (3), several, in particular a plurality corresponding to the plurality of force measurement sensors (5), of elastic force transmission studs (7) which are fixed to the free surfaces of the force measurement sensors (5), wherein the hoof boot (15) has a hoof boot sole (15a) which completely encloses the reaction force measuring plate (1) relative to the ground, and wherein the hoof boot (15) has a hoof boot wall (15c) which is formed integrally with the hoof boot sole (15a) or is fixedly connected to the hoof boot sole (15a), wherein the hoof boot wall (15c) encloses the reaction force measuring plate (1) and, in use, the hoof (H), at least partially, preferably at least substantially.

The invention relates to a hoof boot with a reaction force measuring plate for determining the distribution of reaction forces over a surface when the hoof of a hoofed animal strikes the ground.

For the gait and health analysis of a horse or other hoofed animal (e.g., camel), it is desirable to measure the reaction forces generated when it strikes the ground, not only at a single point, but with a certain displacement across the entire contact area of the hoof. The measured values should enable conclusions to be drawn about the animal's state of health, e.g., with regard to lameness or strain on the musculoskeletal system.

While there are a whole range of solutions that are available for corresponding tasks in the fields of medical diagnostics, training condition analysis and rehabilitation in humans, the availability of corresponding systems for hoofed animals is limited. There is a commercially available product from Tekscan and other systems with a similar purpose, however these are based on measuring acceleration rather than reaction forces.

GB 2 482 192 B discloses the attachment of force sensors to a horseshoe for such purposes and the local storage of their signals and/or their transmission via a wireless transmitter to a remote receiving and evaluation station.

According to DE 10 2011 016 344 A1 , force sensors are used in an elastomer body for a corresponding purpose. The elastomer body, which in turn is to be inserted into a hoof boot, is designed to enable the use of commercially available resistive force sensors with their limited force measuring range for reaction force analysis in horses with their relatively high ground pressure.

From US 2020/319044 A1 we learn of another system for the area-dissipated recording of reaction forces on the hooves of a hoofed animal, which transmits the sensor signals via a wireless transmitter to a remote evaluation unit. This system has a complex design consisting of a base plate and a ground plate, which are guided precisely in relation to each other and between which several force application cylinders and a plurality of resistive force measurement sensors are housed, as well as a microprocessor unit, a communication unit and a battery to supply force to the components. The function of the ‘force application cylinders’ is not apparent from the printed material, nor is the method of attachment to the hoof.

DE 10 2021 211 795.3 (unpublished) describes a reaction force measuring plate for recording the distribution of ground reaction forces across the ground contact area of the foot of a hoofed animal or the foot of a human being when it strikes the ground, with a rigid carrier plate with a first surface facing the ground during use and a second surface facing the hoof or foot, a plurality of flat force measurement sensors fixed in position on the first surface of the carrier plate, and several elastic force transmission studs, especially a plurality corresponding to the plurality of force measurement sensors, fixed to the free surfaces of the force measurement sensors.

A disadvantage of the known possibilities for measuring the force exerted by the foot of a hoofed animal or the foot of a human being when it strikes the ground, i.e. when it comes into contact with the ground, is that only the distributions of force can be measured, however no total forces occur, as shunt forces occur in addition to the discrete sensor positions.

A disadvantage of using individual force transmission studs for each force measurement sensor of the reaction force measuring plate of DE 10 2021 211 795.3 is that even a slight misalignment between the force transmission studs and the force measurement sensor during operation causes the characteristics or calibration curve of the force measurement sensor (measured value vs. applied force) to change. This can result in significant inaccuracies in the measured force.

Another disadvantage is that different hoof shapes and/or ground conditions, for example, can lead to an undefined transmission of force and force dissipation. This can result in shunt forces, which prevent the load from being conducted to the discrete sensor positions as desired.

Another disadvantage is that when used on hoofed animals with iron shoes (e.g., horses), irregularities in the shoeing, e.g., due to protruding nails, can occur, which can greatly influence the distribution of force on a rigid plate. This would not be the case in the reference state without a measuring system on soft ground, as small unevennesses in the ground can sink in and thus be compensated for. An incorrect interpretation of the measurement results could be the case.

One object of the present invention is to improve the possibilities for measuring the force exerted by the foot of a hoofed animal when it strikes the ground. In particular, a preferably compact sensor plate with discrete sensor positions and the transmission of force into the same is to be created, resulting in stable and robust sensor characteristics. Additionally or alternatively, the transmission of force without a shunt force should be used to enable the measurement of absolute forces and minimize potential errors in the measured force distribution. In any case, this should be done in a way that is as simple, cost-effective, space-saving and/or weight-saving as possible. At the very least, an alternative to the familiar options should be created.

According to the invention, the object is achieved by a hoof boot with the features of the independent patent claims. Advantageous further embodiments are described in the subclaims.

The invention therefore relates to a hoof boot with a reaction force measuring plate for detecting the distribution of ground reaction forces across the ground contact area of the foot of a hoofed animal when it strikes the ground, with a preferably rigid carrier plate with a first surface area facing the hoof during use and an opposite second surface area facing the ground, a plurality of flat force measurement sensors fixed in position on the first surface area of the carrier plate, several, in particular a plurality corresponding to the plurality of force measurement sensors, of elastic force transmission studs fixed to the free surfaces of the force measurement sensors, wherein the hoof boot has a hoof boot sole which completely encloses the reaction force measuring plate relative to the ground, and wherein the hoof boot has a hoof boot wall which is formed integrally with the hoof boot sole or is fixedly connected to the hoof boot sole, wherein the hoof boot wall surrounds the reaction force measuring plate and, in use, at least partially, preferably at least substantially, surrounds the hoof.

The invention is therefore based on the idea of forming the desired measuring device of a hoof boot with a base plate that is substantially both flexurally rigid and incompressible (but possibly elastically flexible) carrier plate, to which a plurality of force measurement sensors are attached on one surface area, and whose other surface area is placed on the underside of the hoof during use and in any case is in effective contact with the underside of the hoof.

Furthermore, the invention includes the idea of assigning a force transmission stud adapted to the effective area of the sensor to each of the force measurement sensors on its free surface.

In principle, it is also possible that the system is configured using force transmission studs with a larger base area, which are not assigned to a single force sensor but to two or more force measurement sensors together, and which transfer the ground reaction forces that occur into these sensors. In extreme cases, the measuring plate could even have only one single force transmission stud, with a base area covering all of the force measurement sensors, provided that this force measurement stud, due to its structure and mounting on the carrier plate, enables differentiated transmission of force to the various sensors.

For common practical applications, a number of 3-8 force measurement sensors, in particular 4-6 sensors, is currently considered sufficient. Preferably, at least five force measurement sensors can be used. Preferably, at least, and particularly preferably exactly seven force measurement sensors can be used, which can be arranged along the edge. In any case, the force measurement sensors can be arranged at equal distances from each other in the circumferential direction. This can enable representative recording of force values with limited cost.

Such a reaction force measuring plate has a simple design and is therefore inexpensive to manufacture, robust in practical use, and easy to handle. It also enables a sufficiently resolved recording of the reaction forces across the surface of the hoof with low leakage of the force and therefore high accuracy. It is also flat and lightweight in terms of structure, making it not only easy to use but also easy to transport.

In any case, the corresponding properties and advantages can be integrated into a hoof boot. For this purpose, the reaction force measuring plate can be fixedly arranged inside a hoof boot and thus come into contact with the underside of the hoof of the hoofed animal during use.

The sole of the hoof boot can be formed in one piece, i.e., integrally, in a largely semi-oval shape with the side wall of the hoof boot and can position and guide the reaction force measuring plate laterally. The hoof boot wall or collar can preferably comprise at least ¾ of the sole and preferably be formed completely circumferential. In any case, the side wall of the hoof boot, i.e., the hoof boot wall, can have at least the height of the reaction force measuring plate. The sole of the hoof boot, i.e., the hoof boot sole, can preferably be formed largely in a semi-oval shape with one or more bulges to prevent rotation of one or more of the elements of the reaction force measuring plate. This can preferably be done in the form of two “fins” in the heel area.

According to one aspect of the invention, the hoof boot also has an electronic hoof component which is electrically conductively connected to the force measurement sensors of the reaction force measuring plate by means of a connector element, wherein the connector element passes through a connector element opening in the hoof boot wall, preferably directly above the hoof boot sole and/or laterally. The electronic hoof component may comprise electronics for data acquisition, data processing, for example for signal filtering and/or data handling or data preprocessing, as well as means for data storage and/or data transmission, preferably wireless. It may also store electrical energy to supply the electronics.

In any case, it may be advantageous to route a corresponding connection for data, signal, and/or energy transmission through the connector element opening in the hoof boot wall. Thus, a sensor output from the reaction force measuring plate near the sole base can be routed through an opening in the outer area of the hoof boot. This can protect the connection from stress, in particular from impact on the ground.

Preferably, the sensor output can be routed laterally out of the shoe through an opening in the side wall, which can provide further relief or protection compared to an assembly of the opening at the tip of the hoof. In any case, the sensor output can be routed out on the outer side of the shoe (right hoof boot, right output, left hoof boot, left output) to prevent contact between the connection and, in particular, the electronic hoof component with the other hoof. In any case, the electronic hoof component can be connected to the force measurement sensors as an electronic unit and positioned laterally on the outside of the hoof boot.

According to a further aspect of the invention, the connector element opening furthermore is at least partially, preferably completely, closed by a connector element clamp. This can prevent or at least hinder the entry of dirt and liquid into the interior space of the hoof boot in the area between the underside of the hoof and the reaction force measuring plate at this point. In any case, external influences or environmental influences such as dirt, mud, moisture or water, small stones and the like can be kept away from the force measurement sensors in order to ensure their functionality or improve their longevity.

According to a further aspect of the invention, the electronic hoof component is arranged on the outside of the hoof boot wall, whereby the hoof boot has a hoof component guard which surrounds the electronic hoof component at least from below, preferably also at least on one side, and particularly preferably on both sides. The hoof component guard can be a single part, part of the hoof boot wall, or part of the electronic hoof component itself.

Thus, a holder can be provided for the electronic hoof component, which covers the cable and partially covers the electronics, protecting them from damage and/or fixing them to the outer wall of the shoe. Furthermore, a protective edge can be formed from the sole and/or the wall, which at least partially surrounds the holder and thus protects it from impact and dirt. Alternatively, the holder can be manufactured as part of the sole, which is subsequently folded up and attached to the outer wall of the shoe. Preferably, the hoof component guard can be formed in one piece with the electronic hoof component, preferably its housing.

According to a further aspect of the invention, the hoof boot wall facing away from the carrier plate has a sole profile. This can improve sure-footedness on the ground.

According to a further aspect of the invention, the hoof boot also has an inner protective element which runs parallel to the carrier plate and encloses at least the force measurement sensors opposite the hoof. This provides better protection for the force measurement sensors against dirt and moisture.

According to a further aspect of the invention, the inner protective element has a seal on its edge which seals against the inside of the hoof boot wall. This can improve the protective effect.

According to a further aspect of the invention, the hoof boot also has at least, preferably exactly, one rigid load distribution plate, which is arranged parallel to the carrier plate and facing away from the carrier plate on the elastic force transmission studs and facing the hoof. Thus, the rigid load distribution plate spans several to all force measurement sensors or the distances between them, so to speak. This allows transmission of force per force measurement sensor without shunt forces, which can improve the quality of force measurements.

Preferably, the rigid load distribution plate can be made of a material with very high strength, rigidity and good maximum elongation. Polyamide, with an additional reinforcement can be used for this purpose. The rigid load distribution plate can be implemented as an organic sheet metal made of polyamide with glass fiber reinforcement, which is particularly preferred.

According to a further aspect of the invention, the hoof boot also has at least one, preferably elastomeric, force transmission surface element, which is fixed in position parallel to the carrier plate and facing away from the first surface on the force measurement sensors, whereby the elastic force transmission studs are fixed on the free surface area of the force transmission surface element directly opposite one of the force measurement sensors. The force transmission surface element is also arranged between the carrier plate and the load distribution plate.

This aspect of the present invention is based on the realization that the force transmission studs, which are each arranged directly opposite one of the force measurement sensors, can be used to transmit the ground contact forces to the respective force sensors, as described in DE 10 2021 211 795.3. In addition, however, the fact that a force transmission surface element accommodates the force transmission studs and thus positions them relative to each other and to the force measurement sensors means that any misalignment between the force transmission studs and the force measurement sensor can be avoided during operation, or at least sufficiently reduced in order to avoid and/or at least sufficiently reduce a change and/or an influencing of the characteristics and/or the calibration characteristic curve in order to be able to measure the forces with sufficient accuracy.

The force transmission surface element can also be called a pressure conduit element. In any case, the force transmission surface element can be designed as a thin flat plane, which can preferably be bonded to a carrier film by material engagement, especially by vulcanization, as will be described in more detail below.

The force transmission surface element can also serve as a thin protective layer and, for this purpose, be designed in such a way, that the force transmission surface element covers and protects at least most of the force measurement sensors or their sensor carrier film.

According to a further aspect of the invention, the force transmission studs and/or the force transmission surface element are made of an elastomeric material. This can cause and/or enable elastic force transmission. Especially, the hardness of the elastomer or elastomeric material of the force transmission studs and/or the force transmission surface element can be between 30 and 85 ShA.

According to a further aspect of the invention, the force transmission studs are formed integrally with the force transmission surface element. Thus, the force transmission studs and the force transmission surface element are formed integrally. This can be done using the same material or different materials, although the latter can increase both the manufacturing costs and the design options.

According to a further aspect of the invention, the force transmission studs have a linear or trapezoidal contact surface with the ground and/or with the respective force measurement sensor. Preferably, a linear or elongated contact surface on the side facing away from the force measurement sensor and a larger, wider contact surface in the direction of the force measurement sensor are to be preferred. This can be achieved, for example, by using a trapezoidal shape, but also by using a straight-line design for the height of the force transmission studs. In any case, this can affect force transmission.

According to a further aspect of the invention, the force transmission surface element is bonded to the force transmission studs with the force measurement sensors or a sensor carrier film facing away from the force transmission studs. This can represent a connection option that can be made directly between force measurement sensors and force transmission surface elements. If the force measurement sensors are arranged on a sensor carrier film, and especially, printed on it, as will be described in more detail below, the sensor carrier film can also be covered and protected by means of the force transmission surface element.

According to a further aspect of the invention, the force transmission surface element is vulcanized onto a carrier film away from the force transmission studs, and the carrier film is bonded to the force measurement sensors or a sensor carrier film. The carrier film can thus be used to create a surface that improves the adhesive effect on the force measurement sensors and/or a sensor carrier film. The carrier film may preferably consist of a material chemically similar to the sensor carrier film in order to enable or promote a material bond with the sensor carrier film. In any case, the carrier film may preferably consist of a thermoplastic or elastomeric material (e.g., TPE, NR, EPDM), which can be bonded to the sensor carrier film by material engagement, preferably in a vulcanization process or in an injection molding process.

According to a further aspect of the invention, in geometric configurations adapted for use with various ungulates or other vertebrates (including humans), the carrier plate has the shape of a closed horseshoe, circular ring, U, or a polygon with a recess in the central area.

According to a further aspect of the invention, in a technologically advantageous embodiment, the force measurement sensors, together with associated sensor signal lines and optional power supply lines, are implemented on a continuous sensor carrier film which is fixed to the first surface of the carrier plate.

According to a further aspect of the invention, the force measurement sensors or the sensor carrier film are bonded to the carrier plate and/or the force transmission studs are bonded to the force measurement sensors. This may represent an easily realizable embodiment of the force measurement sensors or the above-mentioned sensor carrier film with the carrier plate.

According to a further aspect of the invention, the effective area of the force measurement sensors is in the range between 0.5 cm² and 10 cm² , in particular 2 cm² and 5 cm². It is understood that when using a relatively large number of sensors, and especially in configurations of the measuring plate intended for animals with a relatively small hoof area, the effective area may be relatively small, while in configurations with a relatively small number of sensors and for animals with a large detection areas, it may be closer to the preferred upper limit or even above it.

According to a further aspect of the invention, in the interest of the technologically easy and cost-effective manufacturability of the sensors and configurability of different embodiments of the measuring plate, all force measurement sensors substantially have the same geometric shape and effective area.

According to a further aspect of the invention, the force measurement sensors are resistive dielectric sensors, which in particular comprise a first conductive layer, a dielectric layer on top of this, which is surrounded and delimited by a spacer determining the shape of the force measurement sensor, and a second conductive layer on the dielectric layer and the spacer. In addition to the structure mentioned here as a variant, such resistive force sensors can also have a different, i.e., known structure. In principle, piezoelectric, capacitive, or inductive sensors or electroactive polymers can also be used in the reaction force measuring plate in addition to resistive dielectric sensors.

According to a further aspect of the invention, material-related embodiments may provide that the carrier plate is made of organic sheet metal, spring steel, or plastic, and/or the force transmission studs are made of elastomer, and/or the load distribution plate is made of organic sheet metal, spring steel, or plastic, preferably polyamide with a strength carrier, preferably glass fiber reinforcement. Specifically, the hardness of the elastomer of the force transmission studs can be between 30 and 85 ShA.

The advantages and practicalities of the invention are also apparent from the description of embodiments based on the figures. These show:

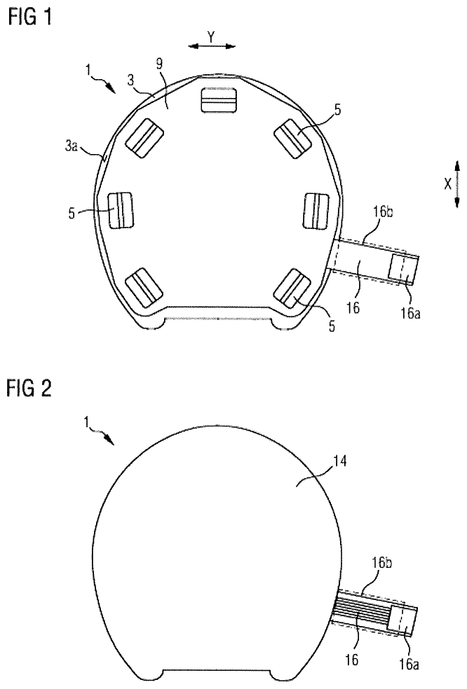

FIG. 1 the structure of an exemplary reaction force measuring plate in a perspective top view of the elastomeric force transmission surface element;

FIG. 2 a top view of the inner protective element of the reaction force measuring plate;

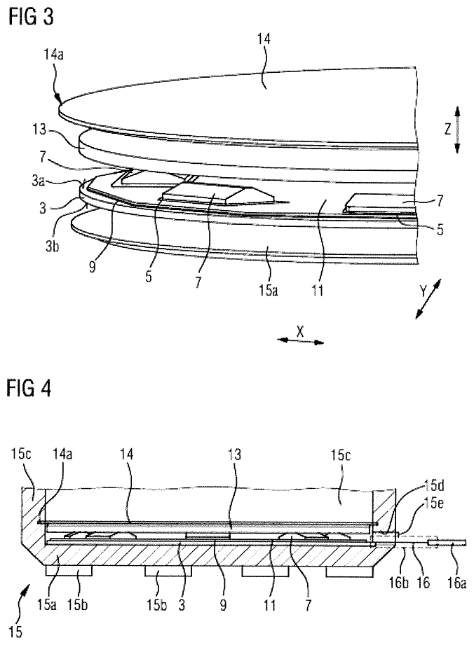

FIG. 3 a perspective view of a section of FIG. 2 as an exploded view with an underside of a hoof boot according to the invention;

FIG. 4 a cross-section through the hoof boot with the reaction force measuring plate of FIGS. 1 to 3;

FIG. 5 a side view of the hoof boot including the electronic hoof component;

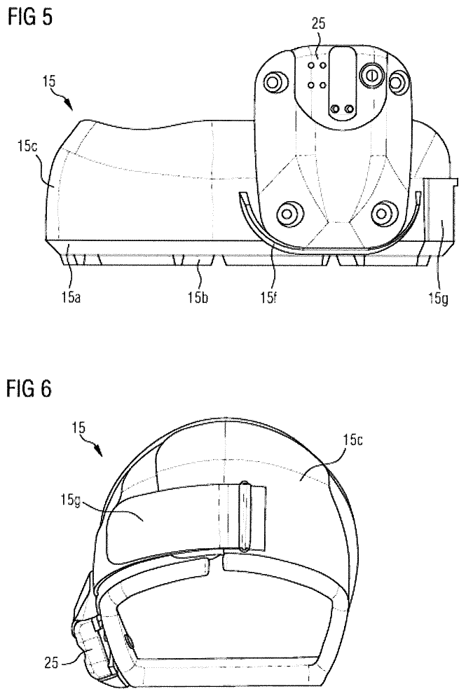

FIG. 6 the representation of FIG. 5 directly from the front; and

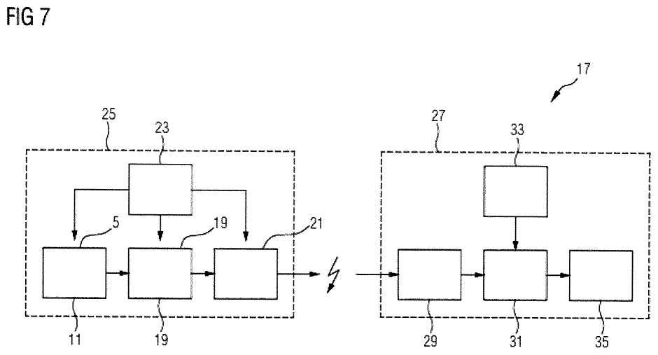

FIG. 7 a schematic diagram of an embodiment of the reaction force measuring system according to the invention, in the form of a block diagram.

The above figures are described in Cartesian coordinates with a longitudinal direction X, a transverse direction Y perpendicular to the longitudinal direction X, and a vertical direction Z perpendicular to both the longitudinal direction X and the transverse direction Y. The longitudinal direction X can also be referred to as depth X, the transverse direction Y as width Y, and the vertical direction Z as height Z. The longitudinal direction X and the transverse direction Y together form the horizontal X, Y, which can also be referred to as the horizontal plane X, Y. The longitudinal direction X, the transverse direction Y, and the vertical direction Z can also be referred to collectively as spatial directions X, Y, Z or as Cartesian spatial directions X, Y, Z.

FIG. 1 shows, in a perspective view from below, the structure of an exemplary reaction force measuring plate 1 with a closed horseshoe-shaped rigid carrier plate 3, which has a first surface 3a and a second surface 3b. Seven resistive force measurement sensors 5 with a matching rectangular basic shape are attached to the first surface 3a at equal distances from each other. The free surface of each of the force measurement sensors 5 faces downwards towards a horse's hoof H, see FIG. 8, and thus away from the ground (not shown). The force measurement sensors 5 can also be referred to as force sensors 5 or force sensor elements 5.

The force measurement sensors 5 are integrally implemented on a sensor carrier film 9, which also carries sensor signal lines as conductors (not shown) for connecting each sensor. The sensor carrier film 9, together with the force measurement sensors 5 formed thereon, can be manufactured using conventional printed circuit board technology known to person skilled in the art. The sensor carrier film 9 is applied with its rear side to the first surface 3a of the carrier 3. This is done using an adhesive and/or an adhesive layer, such as double-sided adhesive tape.

There is also an elastomeric force transmission surface element 11 which, like the sensor carrier film 9, corresponds approximately to the surface area of the rigid carrier plate 3. The elastomeric force transmission surface element 11 can also be referred to as an elastomeric pressure conduit element 11. The elastomeric force transmission surface element 11 has a carrier film 15 facing the rigid carrier plate 3 or the sensor carrier film 9, which is connected to the force transmission surface element 11 by vulcanization and also corresponds approximately to the surface area of the rigid carrier plate 3. The force transmission surface element 11 is also bonded to the sensor carrier film 9 by means of the carrier film (not shown), which is facilitated or achieved by the corresponding material combination. At the same time, the vulcanized bond between the carrier film and the force transmission surface element 11 allows an elastomeric material to be used for the force transmission surface element 11 and still be bonded to the sensor carrier film 9 by means of the carrier film.

Several elastomeric force transmission studs 7, which can also be referred to as elastomeric pressure conduit studs 7, are formed on the elastomeric force transmission surface element 11, which point toward the substrate or away from the force measurement sensors 5 or the sensor carrier film 9. Each force measurement sensor 5 is assigned exactly one force transmission stud 7, so that the force measurement sensor 5 and its force transmission stud 7 are designed to be congruent and positioned relative to each other. This positioning is ensured by the fact that the force transmission studs 7 are formed integrally with the elastomeric force transmission surface element 11.

A rigid load distribution plate 13 is arranged along the vertical axis Z away from the force transmission surface element 11 and parallel to it in the horizontal X, Y, which rests against the force transmission studs 7. This allows force to be transmitted from the ground via the force transmission studs 7 to the rigid load distribution plate 13, enabling the loads to act on the force measurement sensors 5 without any force leakage. The rigid load distribution plate 13 can be implemented as an organic sheet metal made of polyamide with glass fiber reinforcement.

According to the invention, the reaction force measuring plate 1 is arranged and/or integrated within the interior space (not designated) of a hoof boot 15, which is made of an elastomeric material or a material with elastomeric properties, as for example TPE, TPU, PU, and the like, encloses the carrier 3 in a planar, parallel manner on this underside as a hoof boot sole 15a, see FIG. 3, and further in the vertical direction Z from the edge and/or from the border of the carrier 3 via the sensor carrier film 9, the force transmission surface element 11 and the rigid load distribution plate 13 at the edge side upwards (not shown in FIG. 3). The hoof boot sole 15a has a sole profile 15b facing downwards away from the reaction force measuring plate 1. The latter area represents a hoof boot wall 15c, see FIG. 4. This closes off the sensor carrier film 9, including the force measurement sensors 5, and the force transmission surface element 11, including the force transmission studs 7, to the outside and thus protects them from external influences.

Furthermore, an inner protective element 14 is provided, which adjoins the inside of the hoof boot wall 15c in the opposite direction by means of a seal 14a in the form of a sealing lip 14a and encloses and protects the carrier 3 on the back as described above.

The force measurement sensors 5 are connected in printed form by means of the sensor signal lines and can be read out, as will be described in more detail below. For this purpose, a connector element 16 in the form of a plug outlet 16 is provided, which ends in a plug 16a. The plug outlet 16 and an internal part of the plug 16a are enclosed by a cable guard 16b, thereby protecting them from external influences.

The plug outlet 16 leads through a connector element opening 15d in the form of a cable opening 15d to the outside and is otherwise completely closed by a connector element clamp 15e in the form of a cable clamp 15e with spring elasticity.

An electronic hoof component 25, which will be described in more detail in FIG. 7, is arranged on the outside of the hoof boot 15, facing away from the other hoof (not shown) in the transverse direction Y, and is connected to the force measurement sensors 5 by means of the plug outlet 16. The electronic hoof component 25 is surrounded downwardly and laterally toward the front and rear in a U-shape by a hoof component guard 15f.

The hoof boot 15 can be opened and closed by means of a hoof boot closure 15g in order to attach the hoof boot 15 to the hoof or remove it.

FIG. 7 is a schematic diagram of a reaction force measuring system 17, which can be formed, especially with a reaction force measuring plate according to one of FIGS. 1 to 4. The representation takes the form of a functional block diagram and is not intended to show the exact design of the system components.

In addition to the force measurement sensors 5 and their sensor signal lines as already shown in FIGS. 1 to 4, the reaction force measuring system 17 also comprises a sensor signal pre-processing unit 19, which is connected to the force measurement sensors 5 via the sensor signal lines and serves to perform preprocessing and formatting of the sensor signals that is advantageous for external signal transmission. On the output side, the sensor signal pre-processing unit 19 is connected to a wireless sensor signal transmitter 21, which can operate according to the Bluetooth standard, for example, but also according to another standard for wireless message transmission, depending on the application. The above-mentioned components are assigned an energy source 23, which in the simplest case is a commercially available primary cell or a rechargeable battery. If a rechargeable battery is used, it can be assigned a charging socket (not shown in the figure) for recharging when installed.

All of the above components are advantageously arranged in the electronic hoof component 25.

When the system is in use, the electronic hoof component 25 is connected wirelessly via the sensor signal transmitter 21 to a sensor signal receiver, evaluation, and display device 27, which is located remotely from the animal being examined. The device 27 comprises a wireless sensor signal receiver 29, which is configured to communicate with the sensor signal transmitter 21 on the electronic hoof component 25 and which feeds the received signals to a signal evaluation unit 31, where they are evaluated in accordance with a program stored in a program memory 33.

Finally, a display unit 35 serves to display the evaluation results, for example for a therapist or trainer. The components of the sensor signal receiver, evaluation, and display device can, for example, be implemented in a notebook, tablet, or smartphone with a suitable evaluation app.

### REFERENCE LIST (PART OF THE DESCRIPTION)

X longitudinal direction; depth
Y transverse direction; width
Z vertical direction; height
X, Y horizontal; horizontal plane
1 reaction force measuring plate
3 carrier plate
3a first surface
3b second surface
5 force measurement sensor
7 (elastomeric) elastic force transmission studs; (elastomeric) elastic pressure conduit studs
9 sensor carrier film
11 (elastomeric) force transmission surface element; (elastomeric) pressure conduit element
13 load distribution plate
14 inner protective element
14a seal; sealing lip
15 hoof boot; outer protective element
15a hoof boot sole
15b sole profile
15c hoof boot wall
15d connector element opening; cable opening
15e connector element clamp; cable clamp
15f hoof component guard
15g hoof boot closure
16 connector element; plug outlet; cable outlet
16a plug
16b cable guard
17 reaction force measuring system
19 sensor signal pre-processing unit
21 wireless sensor signal transmitter
23 energy source
25 electronic hoof component
27 sensor signal, sensor signal receiver, evaluation and display device
29 wireless sensor signal receiver
31 signal evaluation unit
33 program memory
35 display unit

1. A hoof boot with
a reaction force measuring plate for detecting the distribution of ground reaction forces over the ground contact area of the foot of a hoofed animal when it strikes the ground, with
a preferably rigid carrier plate with a first surface facing the hoof during use and an opposite second surface facing the ground,
a plurality of flat force measurement sensors fixed in position on the first surface of the carrier plate,
a plurality of elastic force transmission studs, in particular a plurality corresponding to the plurality of force measurement sensors, which are fixed to the free surfaces of the force measurement sensors,
wherein the hoof boot has a hoof boot sole which completely encloses the reaction force measuring plate relative to the ground, and
wherein the hoof boot has a hoof boot wall which is formed integrally with the hoof boot sole or is fixedly connected to the hoof boot sole,
wherein the hoof boot wall encloses the reaction force measuring plate and, in use, the hoof, at least partially, preferably at least substantially.

2. The hoof boot according to claim 1,
further comprising an electronic hoof component which is electrically conductively connected to the force measurement sensors of the reaction force measuring plate by means of a connector element,
wherein the connector element passes through a connector element opening in the hoof boot wall, preferably directly above the hoof boot sole and/or laterally.

3. The hoof boot according to claim 2,
wherein the connector element opening is otherwise at least partially, preferably completely, closed by a connector element clamp.

4. The hoof boot according to claim 2,
wherein the electronic hoof component is arranged on the outside of the hoof boot wall,
wherein the hoof boot has a hoof component guard which surrounds the electronic hoof component at least from below, preferably also at least on one side, and particularly preferably on both sides,
wherein the hoof component guard is preferably formed integrally with the electronic hoof component, preferably its housing.

5. The hoof boot according to claim 1,
wherein the hoof boot wall facing away from the carrier plate has a sole profile.

6. The hoof boot according to claim 1,
further comprising an inner protective element which runs parallel to the carrier plate and at least encloses the force measurement sensors relative to the hoof.

7. The hoof boot according to claim 6,
wherein the inner protective element has a seal at its edge, which seals against the inner side of the hoof boot wall.

8. The hoof boot according to claim 1,
further comprising at least, preferably exactly, one rigid load distribution plate which is arranged parallel to the carrier plate and facing away from the carrier plate on the elastic force transmission studs and facing the hoof.

9. The hoof boot according to claim 1,
furthermore comprising at least one, preferably elastomeric, force transmission surface element which is fixed in a fixed position to the force measurement sensors parallel to the carrier plate and facing away from the first surface,
wherein the elastic force transmission studs are fixed to the free surface of the force transmission surface element directly opposite one of the force measurement sensors.

10. The hoof boot according to claim 1,
wherein the carrier plate has the shape of a closed horseshoe, circular ring or U or polygon, especially with a recess in the center area.

11. The hoof boot according to claim 1,
wherein the force measurement sensors together with associated sensor signal lines and optionally power supply lines are realized on a continuous sensor carrier film which is fixed in particular on the first surface of the carrier plate.

12. The hoof boot according to claim 1,
wherein the force measurement sensors or the sensor carrier film are bonded to the carrier plate and/or the force transmission studs are bonded to the force measurement sensors.

13. The hoof boot according to claim 1,
wherein the effective area of the force measurement sensors is in the range between 0.5 cm² and 10 cm² , in particular 2 cm² and 5 cm²,.

14. The hoof boot according to claim 1,
wherein all force measurement sensors are substantially rectangular in shape and have the same geometric shape and effective area.

15. The hoof boot according to claim 1,
wherein the force measurement sensors are resistive-dielectric sensors, which in particular comprise a first conductive layer, on this a dielectric layer which is surrounded and delimited by a spacer determining the shape of the force measurement sensor, and on the dielectric layer and the spacer a second conductive layer.
